(19) United States
(12) Patent Application Publication
(10) Pub. No.: US 2026/0062197 A1
(43) Pub. Date: Mar. 5, 2026

(54) Apparatus and Method to automatically pick pill medications from pill containers, deliver to a pill box using vision and sensors for picker guidance and to confirm pill pick up, give alerts if pill containers are empty, order refills, warn users of harmful medication interactions based on AI

(71) Applicant: Govind Singh Buttar, Saratoga, CA (US)

(72) Inventor: Govind Singh Buttar, Saratoga, CA (US)

(21) Appl. No.: 18/825,819

(22) Filed: Sep. 5, 2024

(51) Int. Cl. B65D 83/04; A61J 7/00

(52) U.S. Cl. CPC B65D 83/0481; A61J 7/0076; A61J 2200/70; A61J 2205/10

(57) ABSTRACT

Sorting medications is generally a tedious, repetitive and an error prone task. To make this task easier and accurate, a configurable and scalable pill sorter to sort medication pills for single or multiple users with redundant checks to minimize any errors in pill picking or dropping and that alerts users if any possible harmful medicine interactions is detected is presented.

### BACKGROUND

Apparatus and Method to automatically pick pill medications from pill containers, deliver to a pill box using vision and sensors for picker guidance and to confirm pill pick up, give alerts if pill containers are empty, order refills, warn users of harmful medication interactions based on AI.

The present applications claim priority to the earlier filed provisional application having Ser. No. 63/536,915, and hereby incorporates subject matter of the provisional application in its entirety.

This apparatus offers significant advantages because it is highly configurable and scalable which makes it suitable for use at home, nursing facilities and pharmacies, offers redundant checks to ensure correct medication pill is picked up and checks for harmful medication interactions using artificial intelligence.

### BRIEF SUMMARY

An easy to use, configurable and scalable automated apparatus is provided that makes process of sorting medication pills easier and faultless by avoiding human errors such as missing pills, or taking more than intended quantity, placing pill in wrong container and alerts the user of any possible harmful interaction between medication pills.

### BRIEF DESCRIPTION OF THE DRAWINGS

Some embodiments of the invention are shown and are not limited by the illustrations.

FIG. 1—Shows the top view of the apparatus

FIG. 2—Shows the front view

FIG. 3—Shows the side view

FIG. 4—Shows the enlarged view of a section of FIG. 3

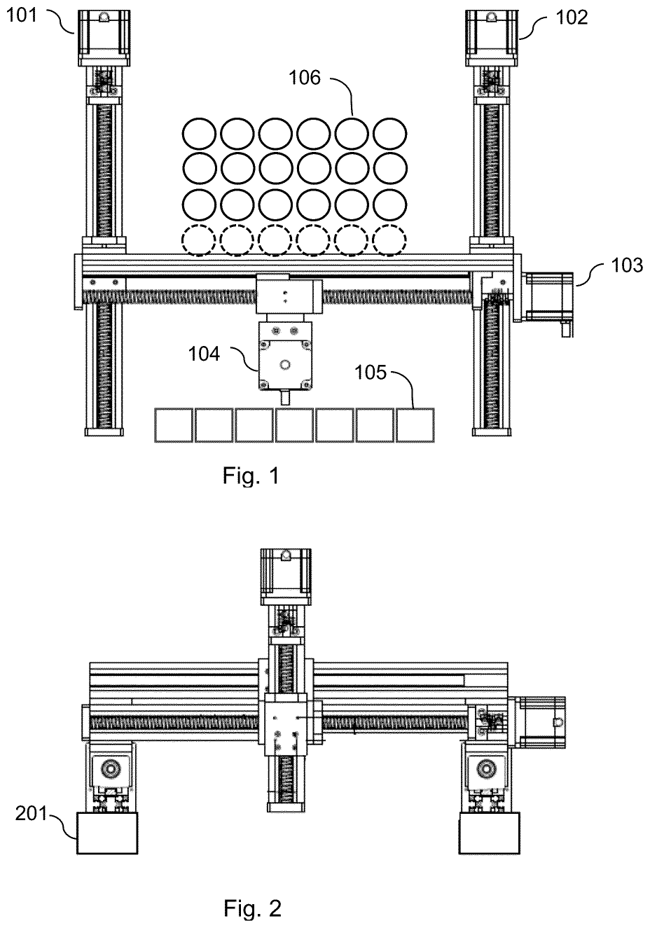

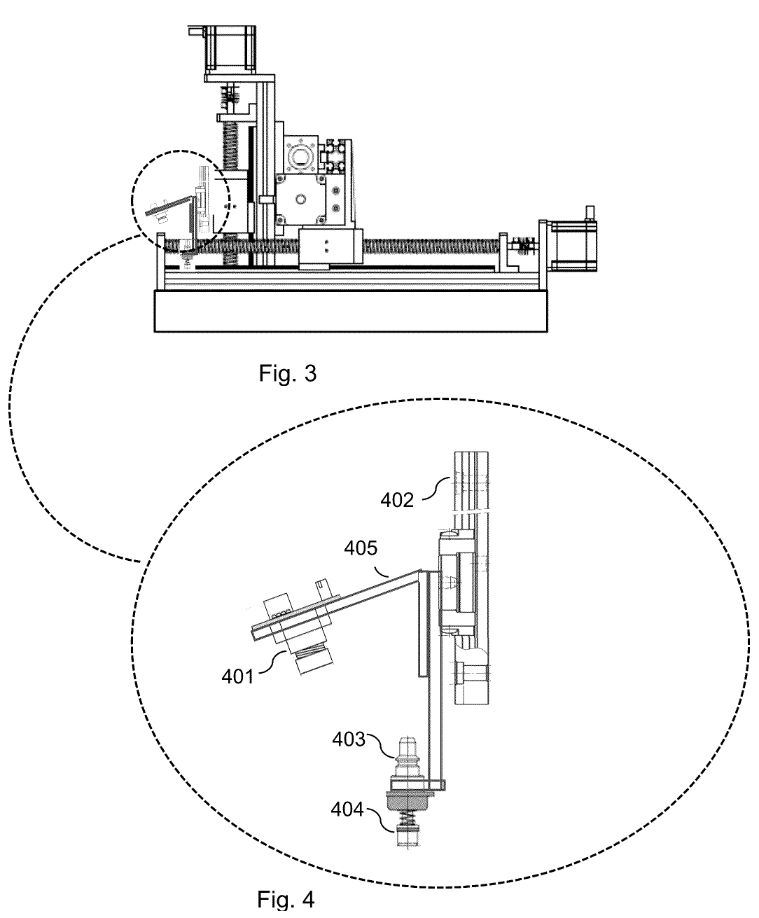

### DESCRIPTION OF COMPONENTS

Positioning system of the apparatus is based on a XYZ stage Gantry Robot. X axis is along the width and Y axis is along the length of the apparatus. The usable length or the stroke length is 15″ that can be customized depending on number and size of pill containers.

Z axis is along the vertical direction of the apparatus. Usable length or the stroke length is 10″ that can be customized depending on vertical height of the pill containers.

The two Y stages are mounted on spacers. The height of the spacers can be changed to accommodate different height of pill containers.

Positioning accuracy of X and Y stages: <0.1 mm.

Pill picker end effector is a spring-loaded vacuum based pick up nozzle. The spring length and stiffness is designed to be compliant that adjusts to the thickness of the pills in the pill container.

Linear slide of 6″ stroke length is installed between the pickup nozzle and the Z-axis stage. Stroke length can be customized depending on maximum fill level of pills in the pill container. The slide is designed to be compliant and moves vertically upwards if the level of the pills in the pill container is high. It moves lower as the pill level in the pill container goes lower and ensures same contact force of picker to the pills at all levels of the pills in the pill container.

Vacuum pump is used to create vacuum for the vacuum nozzle that is used to pick the pills from the pill container using vacuum.

Vacuum sensor is installed in the vacuum line between the vacuum nozzle and the vacuum pump and is used to detect if a pill is picked up from the pill container. A pressure higher than a determined threshold indicates the pill has not been picked up by the vacuum nozzle tip. Similarly, it detects if the pill is successfully dropped at the destination pill box or pill container.

Pneumatic switch: A solenoid controlled pneumatic valve is used to open or close the vacuum line.

Camera is used for: Guiding the vacuum nozzle tip over a pill; Checking if the pill is picked up by the vacuum nozzle tip; detecting the color, shape and size of the pill to check if correct pill is picked up by comparing it to the description of the pill and the location of the pill container stored in a database and to check the level of the pills in the pill container. The user is alerted if the pill quantity is low in the pill container.

Controller board and power supplies: A microcontroller through a software program is used to control the motion of the four linear stages, control the solenoid, read the vacuum sensor, control the pill pickup and drop sequence, send messages to the display to interact with the user, connect to the network and to communicate to an application on a smart phone and order refills.

Application software on smart phone or a personal computer uses Artificial Intelligence to check for possible harmful interaction between medications and alert the user to consult with the pharmacist or medical provider if such interaction is detected.

Display: To communicate to the user by messages on the display.

Quick response code scanner to detect assigned user name and pick selected pills for each user.

### DESCRIPTION OF THE INVENTION

The invention is built on positioning system based on linear stages 101 and 102 in Y-direction, 103 in X-direction and 104 in the Z-direction. The two Y-direction linear stages are each mounted on a spacer 201 the thickness of which can be changed depending on the height of the pill containers used. The configuration automates the picking up the medication pills from but not limited to 100 pills containers locations 106 and placing them in a single cup for the user or transferred to the weekly pill box location 105 or to sort the pills between containers. The apparatus can be run any time the pills need to be taken or run several times to fill a weekly pill box or used to sort pills between containers. The apparatus can be pre-programmed according to the medication schedule to pick up certain medications. The end effector of the pickup tool uses a spring-loaded vacuum tip 404 as a compliant system that auto-adjusts to the different thickness of the pill. The spring-loaded vacuum tip is further mounted on a linear slide to auto adjust to different fill level of pills in the pill container ensuring a constant contact force between the vacuum pick up nozzle and the pills at all pill fill levels in the pill container. The vacuum tool also ensures only one pill is picked up. To ensure a pill is picked up, a vacuum sensor and a camera 401 are used as redundant checks. The vacuum sensor detects drop in pressure when a pill is picked by the nozzle. Similarly, the vacuum sensor detects increase in pressure when a pill is dropped by the nozzle. The camera is mounted on support 405 that is angled to enable clear view of the pills being picked up by the vacuum nozzle. The camera also serves as a visual guide to align the nozzle over a pill inside the pill container. The camera also checks if the pill is dropped in the pill cup or desired pill container. The camera is also used to detect the shape and size of the pill and uses prior saved information to match the pill to the name of the medication as a method to check the correct pill is picked up. The apparatus uses AI to detect for any harmful interaction between the pills. The system is also capable to detect when any pill container is empty and automatically contact the pharmacy or the caregiver to request a refill. The apparatus is able to service up to but not limited to 100 different pill containers. The device also accommodates a quick response code scanner that reads the code assigned to each user and automatically configure the apparatus to fill up medicine cup accordingly or to select pill sorting between containers. The apparatus is highly configurable with redundant checks that make it suitable to be used in home settings by individual users or in a multi user setting such as nursing homes or a pharmacy.

1. A pill sorter comprising: A customizable set of four linear stages in XYZ format to pick pills from up to but not limited to 100 pill containers and deliver them to one pill cup or a weekly pill box or sort the pills between containers; a pill picker; a linear slide; a spring-loaded vacuum nozzle, a vacuum sensor; a camera; a optical scanner, a quick response code scanner; a software that uses artificial intelligence.

2. A said spring-loaded vacuum nozzle capable of picking all sizes/shapes of pills in pill container is attached to the said pill picker.

3. A said linear slide mechanism on which the said picker is attached to accommodate different fill levels of pills in pill container.

4. A said vacuum sensor attached to the said vacuum nozzle to get redundant verification of pill pickup from pill container and drop in target pill box or pill container.

5. A said camera that is attached to the said linear slide and mounted on an angled bracket to view the said picker, to guide the said picker, to get visual confirmation of pill pickup and drop, to get pill detection based on comparing pill size and shape to online database and trigger automatic refills when empty pill container is detected.

6. A said optical scanner to read prescriptions from pill container.

7. A said quick response code scanner to detect assigned user name and automatically configure the apparatus to pick selected pills for each user.

8. A said software that uses artificial intelligence to check for any medication interactions and send alerts to the user.
